(19) United States
(12) Patent Application Publication
(10) Pub. No.: US 2026/0232328 A1
(43) Pub. Date: Aug. 13, 2026

(54) DEVICE AND PROCEDURE FOR ABLATION OF NERVE PAIN FROM THE FACET JOINTS OF THE SPINE

(71) Applicant: INNOVATIVE SURGICAL DESIGNS, INC., Columbus, IN (US)

(72) Inventors: Mark NOVOTNY, Frisco, CO (US); David Greg ANDERSON, Columbus, IN (US); Barry TURNER, Columbus, IN (US); Donna TURNER, Columbus, IN (US)

(21) Appl. No.: 19/534,358

(22) Filed: Feb. 9, 2026

Related U.S. Application Data
(60) Provisional application No. 63/756,596, filed on Feb. 10, 2025.
(60) Provisional application No. 63/870,460, filed on Aug. 26, 2025.

(51) Int. Cl. A61B 17/16

(52) U.S. Cl. CPC A61B 17/1659; A61B 17/1604; A61B 17/1615; A61B 17/1671

(57) ABSTRACT

Provided herein are devices and methods for ablation of a nerve. The devices may include a reamer tool, including a plurality of cutting flutes; a central axis, wherein the device is capable of rotating around the central axis; and a shaft. The methods may include docking a device for ablation of a nerve against a vertebra at a base of a transverse process of the vertebrae; rotating the docked device to cause the plurality of cutting flutes to contact with bone tissue at the base of the transverse process of the vertebrae; and producing a cavity in the bone tissue by the rotating of the docked device, wherein tissue in the cavity is removed and wherein the tissue comprises medial branch nerve.

### CROSS-REFERENCE TO RELATED APPLICATIONS

This application claims the benefit of priority of U.S. Provisional Patent Application No. 63/756,596, filed Feb. 10, 2025, and U.S. Provisional Patent Application No. 63/870,460, filed Aug. 26, 2025. Each application is hereby incorporated by reference in its entirety.

### FIELD

The field of this disclosure relates to the area of pain management and more specifically to the ablation of the medial branch of the dorsal ramus nerve which is the source of pain from the facet joints of the spine.

### BACKGROUND

Chronic back and neck pain are one of the greatest causes of disability and suffering in humans. The costs of treating patients with chronic spinal pain are staggering. Many approaches have been devised by practitioners to address pain including activity changes, medications, procedural interventions and surgery. Unfortunately, none of these approaches has been proven to be universally beneficial and thus the ongoing treatment of chronic spinal pain remains one of the largest unsolved problems in the medical arena presently.

Among the anatomical causes of chronic back and neck pain is pain emanating from the facet joints of the spine. Facet joints are bilaterally paired structures that articulate and allow motion between vertebrae and these structures are subject to arthritic degeneration leading to pain and movement restriction. The facet joints are innervated by a specific sensory nerve called the medial branch of the dorsal ramus. This sensory nerve carries pain signals from the degenerated facet joint back to the central nervous system and thus is a well-known target of clinical therapy. It has been shown that interruption of this sensory nerve is able to diminish or eliminate the pain perception from a degenerated facet joint pain source.

Various modalities have been utilized to treat facet mediated pain including injections of medications such as local anesthetics or steroids into the joint and also the application of energy forms to disrupt the nerve path of pain from the medial branch nerve to the facet joint complex. The most common procedural treatments currently employed involve the use of medial branch nerve interruption or ablation with the use of energy devices placed in close proximity to the medial branch nerve. Examples of energy forms used in these procedures currently include radiofrequency and cryotherapy. These techniques have been able to provide temporary beneficial results by diminishing the medial branch nerve's ability to carry pain signals. Unfortunately, the medial branch nerve will generally regenerate (with time) the ability to carry pain signals after a period of months and will thus result in recurrent pain which may be worse and is generally less successfully treated by reapplication of the energy devices to the nerve at the time of a second treatment of the area. Therefore, many patients continue to suffer increasingly severe back and neck pain symptoms following the temporary relief gained with the current techniques of medial branch nerve ablation.

### SUMMARY

A discovery has been made that provides solutions to at least one or more of the aforementioned problems associated with chronic back and neck pain.

In one embodiment, the present disclosure discloses a mechanical device that is capable of disruption of the nerve supply, for example, to the facet joint complex via the medial branch of the dorsal ramus nerve.

In some aspects, this device is simple to use and provides a quicker and more reliable procedure to ablate the medial branch nerve, for example, based on the fact that the device may rely on bony docking to an anatomically reliable location rather than soft tissue placement.

In some aspects, this device may be inexpensive to produce, package, supply in a sterilized form, inventory, utilized in a clinical procedure and recycle or dispose of after usage.

In some aspects, this device may be able to be provided in a kit form that allows logistical efficiencies in the delivery of this important medical therapy.

In some aspects, this device and technique of nerve ablation may provide a permanent solution due to complete disruption of the nerve pain and blockage of the nerve from regrowing and re-attaching to the facet joint.

In some aspects, this device may be simple for practitioners to learn as it may employ common pre-existing skills that are common to this community of practitioners.

In some aspects, this device may be safer compared to energy forms that are currently used as the potential for malfunction of a complex electronic or electromechanical system is not required.

In some aspects, this device may be cannulated for application over a guide wire which provides for a minimally invasive procedure.

In some aspects, this device may have features to interface with robotic technologies that are now common to spinal procedures.

In one aspect, disclosed is a device for ablation of a nerve, including a reamer tool that may include a plurality of cutting flutes; a central axis, where the device may be capable of rotating around the central axis; and a shaft.

In some aspects, the reamer tool may include a trocar tip. The trocar tip may include a proximal end that may connect with the reamer tool and a sharp distal end.

In some aspects, the trocar tip may include a central axis that may be aligned with the central axis of the device.

In some aspects, the device may include a central cannula. The central cannula may pass through the device along the central axis.

In some aspects, the device may be configured to move along a guide wire. The guide wire may pass through the central cannula.

In some aspects, the plurality of cutting flutes may be arranged in a centrifugal fashion around the central axis of the device.

In some aspects, one or more of the plurality of cutting flutes may be convex-shaped.

In some aspects, one or more components of the device may include a biocompatible material, stainless steel, titanium alloy, cobalt chromium, medical grade plastics and/or biocompatible polymeric materials.

In some aspects, the shaft may be attached to a handle. The handle may be capable of manipulating and controlling a position of the device and applying rotational motion to the device. The handle may include an inner lumen. The inner lumen of the handle may be connected with the central cannula of the device that includes a central cannula. The inner lumen of the handle and the central cannula of the device may form a continuous channel where the guide wire may pass through.

In some aspects, the shaft may include one or more indicators for depth of entry of the trocar tip into a bone.

In some aspects, the one or more indicators may include marks, graduations, numeric features, variations in coloration, and/or a combination thereof.

In another aspect, disclosed is a method for ablation of a nerve, including the steps of: (a) docking any one of the devices disclosed herein against a vertebra at a base of a transverse process of the vertebrae; (b) rotating the docked device to cause the plurality of cutting flutes to contact with bone tissue at the base of the transverse process of the vertebrae; and (c) producing a cavity in the bone tissue by the rotating of the docked device, where tissue in the cavity may be removed. The tissue removed from the cavity may include medial branch nerve. In some aspects, the cavity may be a concavity.

In some aspects, the method may include delivering an implant to the cavity via a guide wire.

In some aspects, the implant may be secured into the cavity with a portion of the implant projecting above a boney surface.

In some aspects, the implant may block path of the medial branch nerve and/or may be capable of blocking growth of the medial branch nerve.

In some aspects, the implant may include an outer threaded surface, optionally with bone threads, and an inner cannula.

In some aspects, the implant may be configured to move along the guide wire. The guide wire may pass through the inner cannula of the implant.

In some aspects, the implant may further include an upper socket. The upper socket may be configured to interface with a driver instrument for implant delivery.

In some aspects, one or more components of the implant may include a biocompatible material, stainless steel, titanium alloy, allograft or xenograft bone, synthetic bone material, medical polymers, polyetheretherketone, carbon fiber, polyetheretherketone reinforced with carbon fiber and/or bioresorbable polymeric materials.

In some aspects, the method may further include localizing a target facet joint using an imaging modality.

In another aspect, disclosed is a system for ablation of a nerve comprising any one of the devices disclosed herein and an implant, where the implant may be capable of blocking nerve growth.

In another aspect, disclosed is a kit for ablation of a nerve including any one of the devices disclosed herein and an implant, where the implant may be capable of blocking nerve growth.

In another aspect, disclosed is a system for ablation of a nerve including any one of the devices disclosed herein and an implant, where the device and related instruments may be configured to interface with a robotic system for anatomically targeting and/or application of a nerve ablation therapy.

In another aspect, disclosed is an implant. The implant may be capable of blocking nerve growth. The implant may be delivered to a cavity via a guide wire. The cavity may be at or in proximity to a base of a transverse process of the vertebrae.

In some aspects, the implant may be secured into the cavity with a portion of the implant projecting above a boney surface.

In some aspects, the implant may block path of the medial branch nerve and/or may be capable of blocking growth of the medial branch nerve.

In some aspects, the implant may include an outer threaded surface, optionally with bone threads, and an inner cannula.

In some aspects, the implant may be configured to move along the guide wire. The guide wire may pass through the inner cannula of the implant.

In some aspects, the implant may further include an upper socket. The upper socket may be configured to interface with a driver instrument for implant delivery. The driver instrument may include an outer wall and a hollow inner shaft. The inner cannula of the implant may be connected with the hollow inner shaft of the driver instrument, both of which may form a continuous channel for a guide wire to pass through. The driver instrument, at its proximal end, may further include a twisting tip. The twisting tip may be connected to the hollow inner shaft. A user may twist or manipulate the twisting tip to rotate the hollow inner shaft, which in turn may cause rotation of the implant at the user's control. The driver instrument disclosed in this paragraph may receive or be connected to any implant disclosed herein.

In another aspect, disclosed is a device for ablation of a nerve, including: a reamer tool, comprising a plurality of cutting flutes; a central axis, where the device is capable of rotating around the central axis; a shaft; and a coring tip, including a proximal end that connects with the reamer tool, a distal end, and a cylindrical wall connecting the proximal end and the distal end, wherein the cylindrical wall defines an inner biopsy chamber, and wherein the inner biopsy chamber has an opening at the distal end.

In some aspects, the coring tip may include a central axis that may be aligned with the central axis of the device.

In some aspects, the device may include a central cannula, where the central cannula passes through the device or portion of the device along the central axis.

In some aspects, the device may be configured to move along a guide wire, where the guide wire passes through the central cannula.

In some aspects, the plurality of cutting flutes may be arranged in a centrifugal fashion around the central axis of the device.

In some aspects, one or more of the plurality of cutting flutes may be convex-shaped.

In some aspects, the coring tip may be configured to remove a cylindrical sample of bone material. The bone material may be capable of blocking nerve growth or regrowth. The bone material may be used to block nerve growth or regrowth.

In some aspects, the coring tip may include one or more side openings in the cylindrical wall to facilitate manual expulsion of the cylindrical sample of bone material from the coring tip upon removal of the device from a body.

In some aspects, the coring tip may have a wall thickness that may be less than ⅛ the diameter of the cylindrical sample of bone material which may be removed in relation to the volume of bone defect created by the use of the device.

In some aspects, the coring tip may include a smooth outer surface to facilitate low friction rotation of the device during usage.

In some aspects, the coring tip may include a sharp distal edge to facilitate cutting of the distal edge of the device into bone tissue during usage.

In some aspects, the coring tip may include cutting teeth along the distal edge to facilitate cutting of hard bony material.

In some aspects, the outer surface of the coring tip may include a threaded profile to facilitate rotatable entry into a bone.

In some aspects, one or more components of the device may include a biocompatible material, stainless steel, titanium alloy, cobalt chromium and/or medical grade plastics.

In some aspects, the shaft may be attached to a handle, and wherein the handle may be capable of manipulating and controlling position of the device and applying rotational motion to the device.

In another aspect, disclosed is an implant for blocking nerve growth, including: a cylindrical shaft with a long axis aligned with the center of the cylindrical shaft with said cylindrical shaft configured to be anchored into tissue; and an upper socket, where the upper socket may be configured to interface with a driver instrument for implant delivery.

In some aspects, the tissue may include bone tissue and/or soft tissue.

In some aspects, the cylindrical shaft may include an outer threaded surface to facilitate anchorage into tissue.

In some aspects, the implant may include a central cannula along the long axis, where the central cannula may allow passage of a guide wire for placement of the implant.

In some aspects, the implant may include a first material within the central cannula, and/or the first material may remain within the central cannula after removal of the guide wire. The first material may include a bone material, a porous ceramic, a bioactive glass, polymeric matrix, a sponge, and/or a foam. The bone material may be autograft bone or allograft bone. The bone material may be capable of blocking nerve growth or regrowth. The bone material may be used to block nerve growth or regrowth. The first material may include pharmacologic agents, medications, biologic molecules, nerve growth modulating agents, anti-inflammatory agents, and/or pain blocking or pain-modulating agents.

In some aspects, the implant may include one or more openings along the outer surface, and/or the one or more openings may be configured to contain a second material. The second material may be the same as the first material. Alternatively, the second material may be different from the first material.

In some aspects, the one or more openings may be at an angle to the long axis of the implant. The angle may be at least, at most, a range of, or exactly 0°, 1°, 2°, 3°, 4°, 5°, 6°, 7°, 8°, 9°, 10°, 11°, 12°, 13°, 14°, 15°, 16°, 17°, 18°, 19°, 20°, 21°, 22°, 23°, 24°, 25°, 26°, 27°, 28°, 29°, 30°, 31°, 32°, 33°, 34°, 35°, 36°, 37°, 38°, 39°, 40°, 41°, 42°, 43°, 44°, 45°, 46°, 47°, 48°, 49°, 50°, 51°, 52°, 53°, 54°, 55°, 56°, 57°, 58°, 59°, 60°, 61°, 62°, 63°, 64°, 65°, 66°, 67°, 68°, 69°, 70°, 71°, 72°, 73°, 74°, 75°, 76°, 77°, 78°, 79°, 80°, 81°, 82°, 83°, 84°, 85°, 86°, 87°, 88°, 89°, 90°, 91°, 92°, 93°, 94°, 95°, 96°, 97°, 98°, 99°, 100°, 101°, 102°, 103°, 104°, 105°, 106°, 107°, 108°, 109°, 110°, 111°, 112°, 113°, 114°, 115°, 116°, 117°, 118°, 119°, 120°, 121°, 122°, 123°, 124°, 125°, 126°, 127°, 128°, 129°, 130°, 131°, 132°, 133°, 134°, 135°, 136°, 137°, 138°, 139°, 140°, 141°, 142°, 143°, 144°, 145°, 146°, 147°, 148°, 149°, 150°, 151°, 152°, 153°, 154°, 155°, 156°, 157°, 158°, 159°, 160°, 161°, 162°, 163°, 164°, 165°, 166°, 167°, 168°, 169°, 170°, 171°, 172°, 173°, 174°, 175°, 176°, 177°, 178°, 179°, or 180°.

In some aspects, the one or more openings may be configured to traverse the implant either along the long axis of the implant or at an angle to the long axis of the implant or both. The angle may be at least, at most, a range of, or exactly 0°, 1°, 2°, 3°, 4°, 5°, 6°, 7°, 8°, 9°, 10°, 11°, 12°, 13°, 14°, 15°, 16°, 17°, 18°, 19°, 20°, 21°, 22°, 23°, 24°, 25°, 26°, 27°, 28°, 29°, 30°, 31°, 32°, 33°, 34°, 35°, 36°, 37°, 38°, 39°, 40°, 41°, 42°, 43°, 44°, 45°, 46°, 47°, 48°, 49°, 50°, 51°, 52°, 53°, 54°, 55°, 56°, 57°, 58°, 59°, 60°, 61°, 62°, 63°, 64°, 65°, 66°, 67°, 68°, 69°, 70°, 71°, 72°, 73°, 74°, 75°, 76°, 77°, 78°, 79°, 80°, 81°, 82°, 83°, 84°, 85°, 86°, 87°, 88°, 89°, 90°, 91°, 92°, 93°, 94°, 95°, 96°, 97°, 98°, 99°, 100°, 101°, 102°, 103°, 104°, 105°, 106°, 107°, 108°, 109°, 110°, 111°, 112°, 113°, 114°, 115°, 116°, 117°, 118°, 119°, 120°, 121°, 122°, 123°, 124°, 125°, 126°, 127°, 128°, 129°, 130°, 131°, 132°, 133°, 134°, 135°, 136°, 137°, 138°, 139°, 140°, 141°, 142°, 143°, 144°, 145°, 146°, 147°, 148°, 149°, 150°, 151°, 152°, 153°, 154°, 155°, 156°, 157°, 158°, 159°, 160°, 161°, 162°, 163°, 164°, 165°, 166°, 167°, 168°, 169°, 170°, 171°, 172°, 173°, 174°, 175°, 176°, 177°, 178°, 179°, or 180°.

In some aspects, the second material may directly or indirectly contact tissues surrounding the implant after placement. The second material may indirectly contact tissues through bodily fluid, tissue fluid, or interstitial fluid.

In some aspects, the second material may include a bone material, a porous ceramic, a bioactive glass, polymeric matrix, a sponge, a foam, a bioactive molecule such as a growth factor or cytokine, a pharmaceutical agent, and/or a combination of these materials. The bone material may be autograft, allograft or xenograft bone. The second material (e.g., a bone material) may be capable of blocking nerve growth or regrowth and/or modulating the pain response in association to the local nerve tissues. The second material (e.g., a bone material) may be used to block, slow, retard, reduce, redirect, and/or prevent nerve growth or regrowth. Additionally, the second material (e.g., a bone material) may act to reduce the ability of the local nerve tissue to produce or transmit pain signals thus reducing the patient's discomfort or suffering.

In some aspects, the second material may include pharmacologic agents, medications, biologic molecules, cytokines, nerve growth modulating agents, anti-inflammatory agents, and/or pain blocking or pain-modulating agents.

In some aspects, the implant may include a base material, with or without a coating material.

In some aspects, the base material may include a biocompatible material, stainless steel, titanium alloy, cobalt chromium, medical grade plastics, bone material, medical grade polymeric compounds, and/or bioresorbable materials. The bone material may be autograft, allograft or xenograft bone.

In some aspects, the bone material may be derived from bone of a subject that receives the implant (e.g., autograft bone) or from bone of a donor (e.g., allograft bone), where the donor may be alive or deceased or from another species (xenograft) or may be synthetically manufactured bone material. The bone material may be capable of blocking nerve growth or regrowth. The bone material may be used to block nerve growth or regrowth.

In some aspects, the coating material may include anti-nerve-growth coating, anti-inflammatory coating, antimicrobial coating, antibacterial coating, antithrombotic coating, anticoagulant coating, hydrophilic coating, hydrophobic coating, drug-eluting coating, growth factor containing, cytokine containing, biocompatible coating, bioactive coating, lubricious coating, anti-adhesion coating, anti-fouling coating, and/or radiopaque coating.

In another aspect, disclosed is a method for blocking nerve growth, including the steps of: disrupting a nerve by producing a cavity in bone tissue; and delivering the implant disclosed herein to the cavity.

In some aspects, the nerve may be a medial branch nerve of the dorsal ramus.

In another aspect, disclosed is method for ablation of a nerve, including the steps of: docking any of the device(s) disclosed herein against a vertebra at a base of a transverse process of the vertebrae; rotating the docked device to cause the plurality of cutting flutes to contact with bone tissue at the base of the transverse process of the vertebrae; producing a cavity in the bone tissue by the rotating of the docked device, wherein tissue in the cavity is removed; and disrupting a medial branch nerve by producing the cavity in the bone tissue.

In some aspects, the method may further include delivering an implant to the cavity via a guide wire, where the implant may include: an outer surface, where a portion of the outer surface may be threaded; a hollow inner channel; and an upper socket, where the upper socket may be configured to interface with a driver instrument for implant delivery.

In some aspects, the outer surface may include one or more openings that may be connected to the hollow inner channel.

In some aspects, the method may further include a core that may be positioned within the hollow inner channel, where at least part of the core may be exposed through the one or more openings.

In some aspects, the core may include a bone material, a porous ceramic, a bioactive glass, a sponge, and/or a foam. The bone material may be autograft, allograft or xenograft bone. The bone material may be capable of blocking nerve growth or regrowth. The bone material may be used to block nerve growth or regrowth.

In some aspects, the implant may include a base material and a coating material.

In some aspects, the base material may include a biocompatible material, stainless steel, titanium alloy, cobalt chromium, medical grade plastics, and/or a bone material. The bone material may be autograft bone or allograft bone. The bone material may be capable of blocking nerve growth or regrowth. The bone material may be used to block nerve growth or regrowth.

In some aspects, the bone material may be derived from bone of a subject that receives the implant (e.g., autograft bone) or from bone of a donor (e.g., allograft bone), where the donor may be alive or deceased or taken from another species (e.g. xenograft) or synthetically manufactured bone.

In some aspects, the coating material may include anti-nerve-growth coating, anti-inflammatory coating, antimicrobial coating, antibacterial coating, antithrombotic coating, anticoagulant coating, hydrophilic coating, hydrophobic coating, drug-eluting coating, biocompatible coating, bioactive coating, growth factor containing, cytokine containing, lubricious coating, anti-adhesion coating, anti-fouling coating, and/or radiopaque coating.

In another aspect, disclosed is a method for ablation of a nerve, including the steps of: docking any of the device(s) disclosed herein against a vertebra at a base of a transverse process of the vertebrae; rotating the docked device to cause the plurality of cutting flutes to contact with bone tissue at the base of the transverse process of the vertebrae; producing a cavity in the bone tissue by the rotating of the docked device, where tissue in the cavity may be removed by the coring tip and captured in the inner biopsy chamber of the coring tip; and disrupting a medial branch nerve by producing the cavity in the bone tissue.

In some aspects, the method may further include: ejecting the removed tissue from the coring tip; loading the removed tissue into an implant; and delivering the implant to the cavity via a guide wire. The removed tissue may include bone tissue. The bone tissue may be capable of blocking nerve growth or regrowth. The bone tissue may be used to block nerve growth or regrowth.

In some aspects, where the implant may include: an outer surface, where a portion of the outer surface may be threaded; a hollow inner channel; and an upper socket, where the upper socket may be configured to interface with a driver instrument for implant delivery; where the outer surface may include one or more openings that may be connected to the hollow inner channel.

In some aspects, the removed tissue may be positioned within the hollow inner channel of the implant, where at least part of the removed tissue may be exposed through the one or more openings. The removed tissue may include bone tissue. The bone tissue may be capable of blocking nerve growth or regrowth. The bone tissue may be used to block nerve growth or regrowth.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The devices and methods for their use can “comprise,” “consist essentially of,” or “consist of” any of the ingredients or steps disclosed throughout the specification. Devices and methods “consisting essentially of” any of the ingredients or steps disclosed limits the scope of the claim to the specified materials or steps which do not materially affect the basic and novel characteristic of the claimed invention.

It is contemplated that any embodiment discussed in this specification can be implemented with respect to any method or device of the disclosure, and vice versa. Furthermore, devices of the disclosure can be used to achieve methods of the disclosure.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A-1I show, in accordance with aspects of the present disclosure, embodiments of exemplary devices for ablation of a nerve including a reamer tool with a trocar tip or a reamer tool having a central cannula, with different views of different parts of the devices, handles that may receive such devices, an exemplary implant, and different views of the exemplary implant; and

FIGS. 2A-2O show, in accordance with aspects of the present disclosure, embodiments of exemplary devices for ablation of a nerve including a reamer tool with a coring tip, with different views of different parts of the devices, handles that may receive such devices, an exemplary implant with one or more openings along its outer surface, a driver instrument that may receive an implant, and different views of the exemplary implant and driver instrument.

### DETAILED DESCRIPTION

### I. Exemplary Device Description

The present disclosure has multiple embodiments. Exemplary embodiments are described, although this should not be taken as a limitation of the various forms that this disclosure could be applied to address the present problem of facet mediated spinal pain.

### A. A Device With a Reamer Tool That Has a Trocar Tip

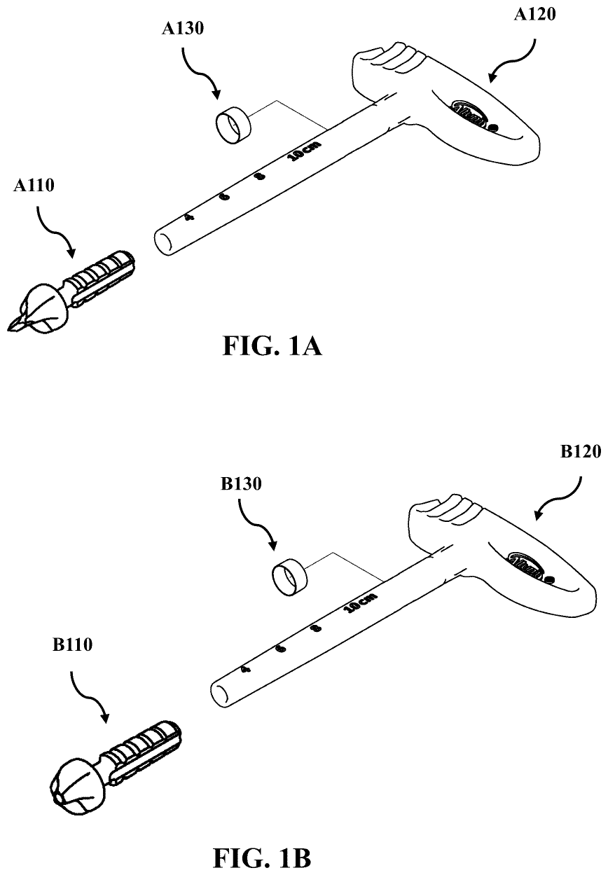

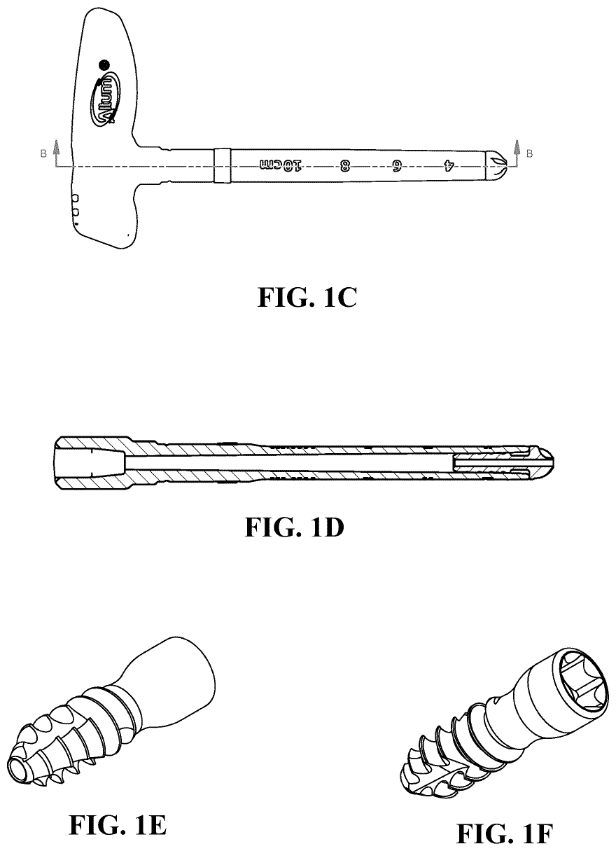

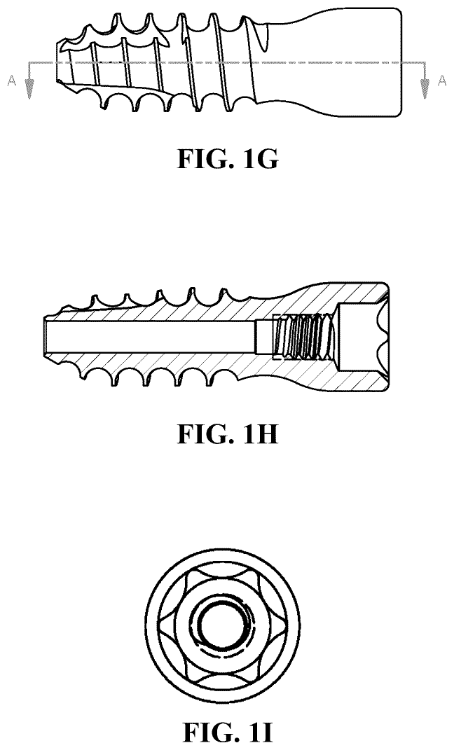

In one embodiment, the device includes a reamer tool (e.g., shown in FIGS. 1A and 1B) for removal of the medial branch nerve tissue which may be used with or without an implantable device that is used to blockade the path of nerve regrowth.

In some aspects, the reamer tool has a trocar tip (e.g., shown in FIG. 1A), a thin projection, that may be sharp at the distal end to engage with and embed a short distance (few millimeters, such as at least, at most, a range of, or exactly 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, or 16 mm) into the bone at the target area (base of the transverse process of the vertebrae). The trocar tip may be aligned with the central axis of the device and may serve as a pivot point for the rotational movement of the device around the central axis of the trocar tip. Just proximal to the trocar tip may be a reaming surface including a multiplicity of cutting flutes arranged in a centrifugal fashion around the central axis of the device. The general shape of the cutting flutes may be convex such that the reamer may make a slightly concave shaped cavity when the reamer is pressed against the bone and rotated to remove tissue at the site of contact. The reamer may be attached to a shaft with a length that is sufficient to allow the device to be placed percutaneously through a small incision to reach the spine. The shaft may be attached to a handle (e.g., shown in FIGS. 1A and 1B) that allows a practitioner to manipulate and control the position of the device and to apply rotational motion to the device in order to achieve the goal of tissue removal along the path of the medial branch nerve supply.

In another embodiment of the reamer, a cannulated version of the reamer (e.g., shown in FIG. 1B) could be employed instead of the trocar tipped reamer. In this embodiment, there would be a central passage through the reamer instrument that would allow the passage of the reamer over a guide wire.

Common biocompatible materials would be used to manufacture the reamer instrument such as stainless steel, titanium alloy, cobalt chromium, medical grade plastics, and/or medical grade polymeric materials.

The disclosure may also include an implant (e.g., shown in FIGS. 1E-1I) that can be placed after the nerve tissue is disrupted to prevent regrowth of nerve tissue in the future.

One embodiment of the implant would be a cannulated cylindrical screw (e.g., shown in FIGS. 1E-1I) which may be threaded a short distance (few millimeters, such as at least, at most, a range of, or exactly 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, 40 mm, 41 mm, 42 mm, 43 mm, 44 mm, 45 mm, 46 mm, 47 mm, 48 mm, 49 mm, or 50 mm) into the bone at the side of the nerve pathway to block regrowth of the medial branch nerve supply. This implant would have an outer threaded surface with bone threads and an inner passage or cannulation that could be used to deliver the implant over a guide wire. The implant would have an upper socket to interface with a driver instrument for implant delivery. The implant could be manufactured from common biocompatible materials such as stainless steel, titanium alloy, allograft bone, synthetic bone material, and/or medical polymers such as polyetheretherketone (also known as polyether ether ketone, polyetherether ketone, or PEEK), and/or polyetheretherketone reinforced with carbon fiber, etc.

### B. a Device With a Reamer Tool That Has a Coring Tip

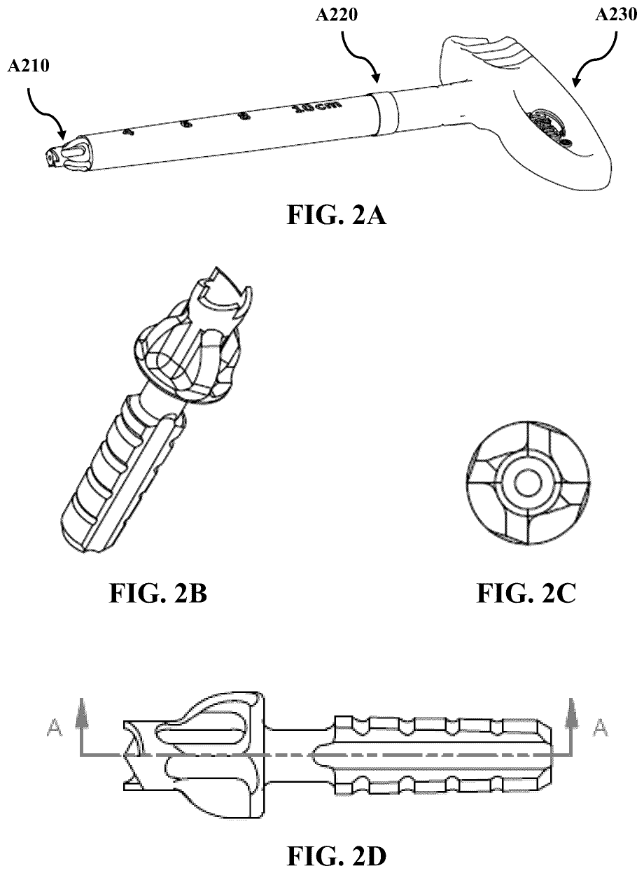

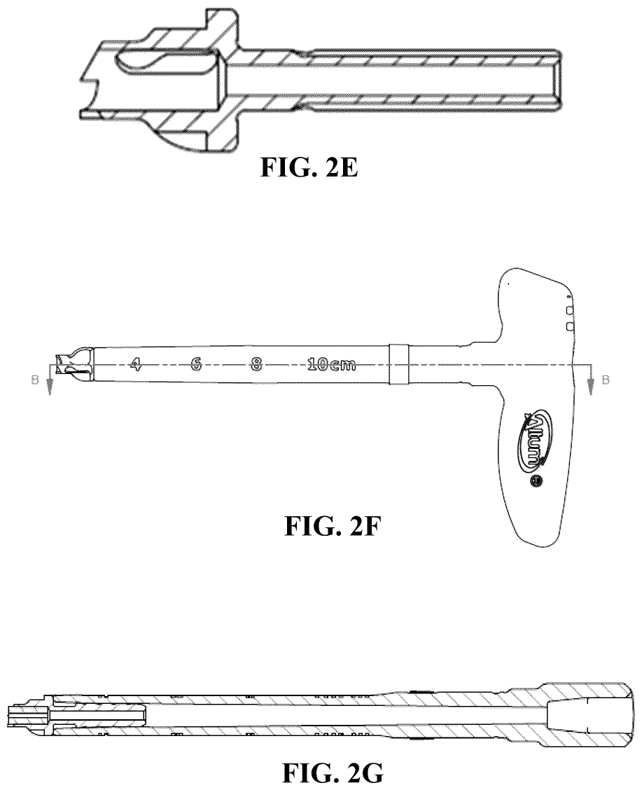

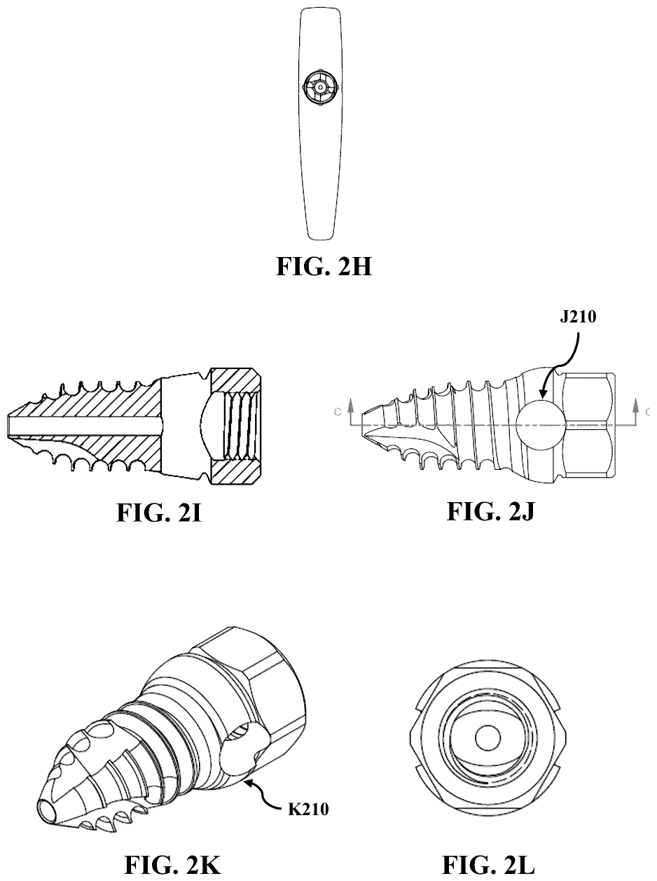

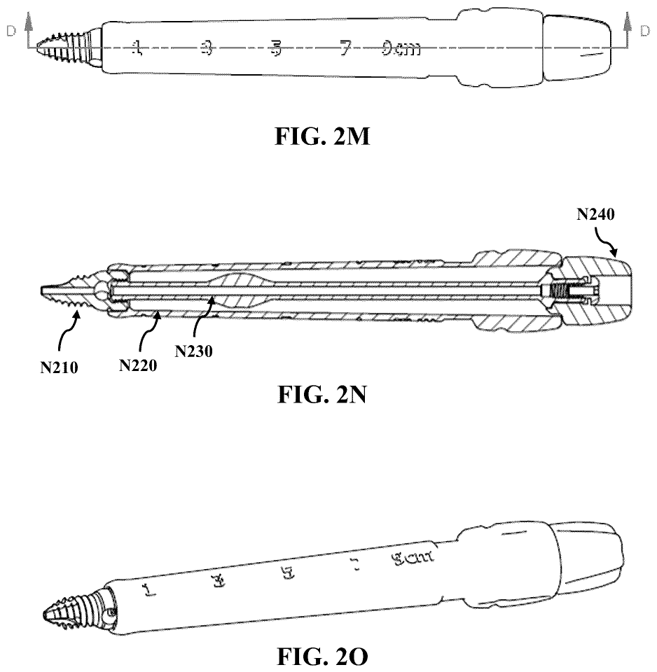

In another embodiment, the device includes a reamer tool (e.g., shown in FIGS. 2A and 2B-E) for removal of the medial branch nerve tissue which may be used with or without an implantable device that is used to blockade the path of nerve regrowth.

In some aspects, the reamer tool has a coring tip (e.g., shown in FIGS. 2A and 2B-E). The coring tip may include a proximal end that connects with the reamer tool, a distal end, and a cylindrical wall connecting the proximal end and the distal end. The cylindrical wall may define an inner biopsy chamber, and the inner biopsy chamber may have an opening at the distal end. The coring tip may have a central axis that may be aligned with the central axis of the device. The coring tip may be aligned with the central axis of the device and may serve as a pivot point for the rotational movement of the device around the central axis of the coring tip. Just proximal to the coring tip may be a reaming surface including a multiplicity of cutting flutes arranged in a centrifugal fashion around the central axis of the device. The general shape of the cutting flutes may be convex such that the reamer may make a slightly concave shaped cavity when the reamer is pressed against the bone and rotated to remove tissue at the site of contact. The reamer may be attached to a shaft with a length that is sufficient to allow the device to be placed percutaneously through a small incision to reach the spine. The shaft may be attached to a handle (e.g., shown in A230 in FIG. 2A) that allows a practitioner to manipulate and control the position of the device and to apply rotational motion to the device in order to achieve the goal of tissue removal along the path of the medial branch nerve supply.

In another embodiment of the reamer, a cannulated version of a reamer having a coring tip (e.g., shown in FIG. 2B-E) could be employed. The device may include a central cannula (e.g., shown in a cross section view in FIG. 2C and in a longitudinal section view in FIG. 2E), where the central cannula passes through the device or portion of the device along the central axis. In this embodiment, there would be a central passage through the reamer instrument that would allow the passage of the reamer over a guide wire.

Common biocompatible materials would be used to manufacture the reamer instrument such as stainless steel, titanium alloy, cobalt chromium, medical grade plastics, and/or medical grade polymeric compounds.

The disclosure may also include an implant (e.g., shown in FIGS. 2I-2L) that can be placed after the nerve tissue is disrupted to prevent regrowth of nerve tissue in the future.

One embodiment of the implant would be a cannulated cylindrical or cone-shaped screw (e.g., shown in FIGS. 2I-2L) which may be threaded a short distance (few millimeters, such as at least, at most, a range of, or exactly 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, 40 mm, 41 mm, 42 mm, 43 mm, 44 mm, 45 mm, 46 mm, 47 mm, 48 mm, 49 mm, or 50 mm) into the bone at the side of the nerve pathway to block regrowth of the medial branch nerve supply. This implant would have an outer threaded surface with bone threads and an inner passage or cannulation that could be used to deliver the implant over a guide wire. The implant would have an upper socket to interface with a driver instrument (e.g., shown as FIGS. 2M-O) for implant delivery. The implant could be manufactured from common biocompatible materials such as stainless steel, titanium alloy, allograft or xenograft bone, synthetic bone material, and/or medical polymers such as polyetheretherketone (also known as polyether ether ketone, polyetherether ketone, or PEEK), and/or polyetheretherketone reinforced with carbon fiber, and/or bioresorbable materials.

One embodiment of the implant may include a central cannula along the long axis, where the central cannula may allow passage of a guide wire for placement of the implant. In some embodiments, the implant may include a first material within the central cannula, and/or the first material may remain within the central cannula after removal of the guide wire.

In some embodiments, the first material may include a bone material, a porous ceramic, a bioactive glass, polymeric matrix, a sponge, and/or a foam. The bone material may be autograft bone or allograft bone. The bone material may be capable of blocking nerve growth or regrowth. The bone material may be used to block nerve growth or regrowth.

In some embodiments, the first material may include pharmacologic agents, medications, biologic molecules, nerve growth modulating agents, anti-inflammatory agents, and/or pain blocking or pain-modulating agents.

In some embodiments, the implant may include one or more openings (e.g., shown as J210 in FIG. 2J and K210 in FIG. 2K) along the outer surface, and/or the one or more openings may be configured to contain a second material. The second material may be the same as the first material. Alternatively, the second material may be different from the first material.

In some embodiments, the one or more openings may be at an angle to the long axis of the implant.

In some embodiments, the one or more openings may be configured to traverse the implant either along the long axis of the implant or at an angle to the long axis of the implant or both.

In some embodiments, the second material may directly or indirectly contact tissues surrounding the implant after placement.

In some embodiments, the second material may include a bone material, a porous ceramic, a bioactive glass, polymeric matrix, a sponge, and/or a foam. The bone material may be autograft bone or allograft bone. The bone material may be capable of blocking nerve growth or regrowth. The bone material may be used to block nerve growth or regrowth.

In some embodiments, the second material may include pharmacologic agents, medications, biologic molecules, nerve growth modulating agents, anti-inflammatory agents, and/or pain blocking or pain-modulating agents.

In some embodiments, the implant may include a base material, with or without a coating material.

In some embodiments, the base material may include a biocompatible material, stainless steel, titanium alloy, cobalt chromium, medical grade plastics, and/or a bone material. The bone material may be autograft bone or allograft bone. The bone material may be capable of blocking nerve growth or regrowth. The bone material may be used to block nerve growth or regrowth.

In some embodiments, the bone material may be derived from bone of a subject that receives the implant (e.g., autograft bone) or from bone of a donor (e.g., allograft bone), where the donor may be alive or deceased. The bone material may be autograft bone or allograft bone. The bone material may be capable of blocking nerve growth or regrowth. The bone material may be used to block nerve growth or regrowth.

In some embodiments, the coating material may include anti-nerve-growth coating, anti-inflammatory coating, antimicrobial coating, antibacterial coating, antithrombotic coating, anticoagulant coating, hydrophilic coating, hydrophobic coating, drug-eluting coating, biocompatible coating, bioactive coating, growth factor containing, cytocytokine containing, lubricious coating, anti-adhesion coating, anti-fouling coating, and/or radiopaque coating.

### II. Exemplary Methods and Uses

In one embodiment, to use the device disclosed herein having a trocar tip, a medical practitioner would first localize the site of the painful facet joint using an imaging modality such as fluoroscopy. Next, a small incision would be made to allow the reamer device to be delivered to the site of the medial branch nerve pathway in a percutaneous or minimally invasive fashion. The reamer device would then be docked against the vertebrae at the base of the transverse process which is the anatomically reliable path for the medial branch nerves. For the embodiment of the reamer device with the trocar tip, the trocar tip would be lightly impacted to cause the trocar tip to embed in the bone at the docking site for a few millimeters, such as at least, at most, a range of, or exactly 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm. Next, the reamer device would be rotated using the handle while applying light downward pressure to cause the reamer cutting flutes to come into contact with the bone tissue at the base of the transverse process. This would cause the reaming surface to remove tissue at the docking site in a cylindrical fashion, creating a slight concavity (up to a few millimeters deep, such as at least, at most, a range of, or exactly 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, or 16 mm) in the bone at the docking site and disrupting the medial branch nerve supply to the facet joints. After performing this nerve ablating procedure, the device would be withdrawn, and the skin site of the small incision would be closed with standard closure methods well known to the art of spinal medical procedures. Additionally or alternatively, the skin site of the small incision may remain open to allow delivery of an implant as described below.

In another embodiment to use the device disclosed herein having a cannulated passage, a medical practitioner would first localize the site of the painful facet joint using an imaging modality such as fluoroscopy. Next, a small incision would be made to allow a targeting needle, such as a Jamshidi needle, to be delivered to the site of the medial branch nerve pathway at the base of the transverse process of the vertebra. After positioning of the targeting needle, the needle tip would be impacted a short distance (few millimeters, such as at least, at most, a range of, or exactly 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm) into the bone at the base of the transverse process. Next, a guide wire would be placed into the bone through the bore in the targeting needle and the needle would be withdrawn while maintaining the position of the guide wire. Next, the reamer device with a cannulated passage would then be docked against the vertebrae at the base of the transverse process which is the anatomically reliable path for the medial branch nerves. Next, the reamer device would be rotated around the axis of the guide wire using the handle while applying light downward pressure to cause the reamer cutting flutes to come into contact with the bone tissue at the base of the transverse process. This would cause the reaming surface to remove tissue at the docking site in a cylindrical fashion, creating a slight concavity (up to a few millimeters deep, such as at least, at most, a range of, or exactly 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, or 16 mm) in the bone at the docking site and disrupting the medial branch nerve supply to the facet joints. After performing this nerve ablating procedure, the device and guide wire would be withdrawn, and the skin site of the small incision would be close with standard closure methods well known to the art of spinal medical procedures. Additionally or alternatively, the skin site of the small incision may remain open to allow delivery of an implant as described below.

In another embodiment, to use an implant, such as a nerve growth blocking implant, the practitioner would use a guide wire, previously placed at the time of cannulated reamer usage (as described above), to place an implant (e.g., a cannulated implant) into contact with the site of the bony concavity formed by the use of the reamer device and embed the implant in a location such that it would be a physical block to the regrowth or the medial branch nerve. The implant would be attached to and manipulated by a cannulated driver instrument to allow the practitioner to tread the implant into the bone at the site of the reamer defect. The practitioner would then apply downward pressure while threadably delivering the device to a depth that would securely anchor the implant into the bone tissue while providing that a portion of the implant projecting above the bony surface to physically block the path of the medial branch nerve and thus to block the potential for nerve regrowth. After radiographic confirmation of the correct localization of the nerve regrowth blocking implant, the driver instrument would be detached from the implant and withdrawn from the patient. The skin incision would be closed using standard techniques commonly known to the art of spinal procedures.

In one embodiment, to use the device disclosed herein having a coring tip, a medical practitioner would first localize the site of the painful facet joint using an imaging modality such as fluoroscopy. The coring tip may be any coring tip disclosed herein, including a coring tip that includes a proximal end that connects with the reamer tool, a distal end, and a cylindrical wall connecting the proximal end and the distal end, where the cylindrical wall defines an inner biopsy chamber, and where the inner biopsy chamber has an opening at the distal end. The coring tip may be configured to remove a cylindrical sample of bone material. The bone material may be capable of blocking nerve growth or regrowth. The bone material may be used to block nerve growth or regrowth.

Next, a small incision would be made to allow the reamer device to be delivered to the site of the medial branch nerve pathway in a percutaneous or minimally invasive fashion. The reamer device would then be docked against the vertebrae at the base of the transverse process which is the anatomically reliable path for the medial branch nerves. For the embodiment of the reamer device with the coring tip, the coring tip would be lightly impacted to cause the coring tip to embed in the bone at the docking site for a few millimeters, such as at least, at most, a range of, or exactly 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm. Next, the reamer device would be rotated using the handle while applying light downward pressure to cause the reamer cutting flutes to come into contact with the bone tissue at the base of the transverse process. This would cause the reaming surface to remove tissue at the docking site in a cylindrical fashion, creating a slight concavity (up to a few millimeters deep, such as at least, at most, a range of, or exactly 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, or 16 mm) in the bone at the docking site and disrupting the medial branch nerve supply to the facet joints. Tissue in the concavity may be removed by the coring tip and captured in the inner biopsy chamber of the coring tip. The tissue or removed tissue may be bone tissue. After performing this nerve ablating procedure, the device would be withdrawn. The tissue captured in the inner biopsy chamber of the coring tip would be retrieved together with the withdrawn device. The removed tissue from the coring tip may be ejected and loaded into an implant disclosed herein. Afterwards, the skin site of the small incision may be closed with standard closure methods well known to the art of spinal medical procedures. Additionally or alternatively, the skin site of the small incision may remain open to allow delivery of an implant as described below.

In some embodiments, the device with the coring tip may include a central cannula. The central cannula may pass through the device or portion of the device along the central axis. The device may be configured to move along a guide wire, where the guide wire passes through the central cannula. A medical practitioner would first localize the site of the painful facet joint using an imaging modality such as fluoroscopy. Next, a small incision would be made to allow a targeting needle, such as a Jamshidi needle, to be delivered to the site of the medial branch nerve pathway at the base of the transverse process of the vertebra. After positioning of the targeting needle, the needle tip would be impacted a short distance (few millimeters, such as at least, at most, a range of, or exactly 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, or 20 mm) into the bone at the base of the transverse process. Next, a guide wire would be placed into the bone through the bore in the targeting needle and the needle would be withdrawn while maintaining the position of the guide wire. Next, the reamer device with the coring tip that includes a central cannula would then be docked against the vertebrae at the base of the transverse process which is the anatomically reliable path for the medial branch nerves. Next, the reamer device would be rotated around the axis of the guide wire using the handle while applying light downward pressure to cause the reamer cutting flutes to come into contact with the bone tissue at the base of the transverse process. This would cause the reaming surface to remove tissue at the docking site in a cylindrical fashion, creating a slight concavity (up to a few millimeters deep, such as at least, at most, a range of, or exactly 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, or 16 mm) in the bone at the docking site and disrupting the medial branch nerve supply to the facet joints. Tissue in the concavity may be removed by the coring tip and captured in the inner biopsy chamber of the coring tip. The tissue or removed tissue may be bone tissue. After performing this nerve ablating procedure, the device would be withdrawn. The tissue captured in the inner biopsy chamber of the coring tip would be retrieved together with the withdrawn device. The removed tissue from the coring tip may be ejected and loaded into an implant disclosed herein. Afterwards, the skin site of the small incision may be closed with standard closure methods well known to the art of spinal medical procedures. Additionally or alternatively, the skin site of the small incision may remain open to allow delivery of an implant as described below.

In another embodiment, to use an implant, such as a nerve growth blocking implant, the practitioner would use a guide wire, previously placed at the time of cannulated reamer usage (as described above), to place an implant (e.g., a cannulated implant) into contact with the site of the bony concavity formed by the use of the reamer device and embed the implant in a location such that it would be a physical block to the regrowth or the medial branch nerve. In some embodiments, the implant may include an outer surface, where a portion of the outer surface is threaded; a hollow inner channel; and an upper socket, where the upper socket is configured to interface with a driver instrument for implant delivery; where the outer surface may include one or more openings that are connected to the hollow inner channel. The tissue removed from the coring tip of the reamer device may be ejected and loaded into the implant within the hollow inner channel of the implant, where at least part of the removed tissue is exposed through the one or more openings. The removed tissue may include bone tissue. The bone tissue may be capable of blocking nerve growth or regrowth. The bone tissue may be used to block nerve growth or regrowth.

The implant would be attached to and manipulated by a cannulated driver instrument to allow the practitioner to tread the implant into the bone at the site of the reamer defect. The practitioner would then apply downward pressure while threadably delivering the device to a depth that would securely anchor the implant into the bone tissue while providing that a portion of the implant projecting above the bony surface to physically block the path of the medial branch nerve and thus to block the potential for nerve regrowth. After radiographic confirmation of the correct localization of the nerve regrowth blocking implant, the driver instrument would be detached from the implant and withdrawn from the patient. The skin incision would be closed using standard techniques commonly known to the art of spinal procedures.

In another embodiment of the current invention, the device would have features allowing it to interface with a medical robotic system capable of accurate targeting of the spinal anatomy. Using either a trocar-tipped or cannulated embodiment of the device, the robotic system will perform or guide the placement of the reamer device into the correct location of the medial branch nerve at the base of the transverse process. Next, the application of rotation and downward pressure would be applied either manually by the supervising practitioner or by the robotic system and tissue removal would occur in similar fashion described above.

Also disclosed in the context of the present disclosure are clauses 1 to 68.

Clause 1: A device for ablation of a nerve, comprising: a reamer tool, comprising a plurality of cutting flutes; a central axis, wherein the device is capable of rotating around the central axis; and a shaft.

Clause 2: The device of clause 1, wherein the reamer tool comprises a trocar tip, and wherein the trocar tip comprises a proximal end that connects with the reamer tool and a sharp distal end.

Clause 3: The device of clause 2, wherein the trocar tip comprises a central axis that is aligned with the central axis of the device.

Clause 4: The device of clause 1, wherein the device comprises a central cannula, wherein the central cannula passes through the device along the central axis.

Clause 5: The device of clause 4, wherein the device is configured to move along a guide wire, wherein the guide wire passes through the central cannula.

Clause 6: The device of any one of clauses 1 to 5, wherein the plurality of cutting flutes are arranged in a centrifugal fashion around the central axis of the device.

Clause 7: The device of any one of clauses 1 to 6, wherein one or more of the plurality of cutting flutes is convex-shaped.

Clause 8: The device of any one of clauses 1 to 7, wherein one or more components of the device comprises a biocompatible material, stainless steel, titanium alloy, cobalt chromium, medical grade plastic, and/or a polymeric compound.

Clause 9: The device of any one of clauses 1 to 8, wherein the shaft is attached to a handle, and wherein the handle is capable of manipulating and controlling a position of the device and applying rotational motion to the device.

Clause 10: The device of any one of clauses 1 to 9, wherein the shaft comprises one or more indicators for depth of entry of the trocar tip into a bone.

Clause 11: The device of clause 10, wherein the one or more indicators comprise marks, graduations, numeric features, variations in coloration, and/or a combination thereof.

Clause 12: A method for ablation of a nerve, comprising the steps of: docking the device of any one of clauses 1 to 11 against a vertebra at a base of a transverse process of the vertebrae; rotating the docked device to cause the plurality of cutting flutes to contact with bone tissue at the base of the transverse process of the vertebrae; and producing a cavity in the bone tissue by the rotating of the docked device, wherein tissue in the cavity is removed and wherein the tissue comprises medial branch nerve.

Clause 13: The method of clause 12, further comprising delivering an implant to the cavity via a guide wire.

Clause 14: The method of clause 13, wherein the implant is secured into the cavity with a portion of the implant projecting above a boney surface.

Clause 15: The method of clause 14, wherein the implant blocks path of the medial branch nerve and/or is capable of blocking growth of the medial branch nerve.

Clause 16: The method of clause 13, wherein the implant comprises an outer threaded surface, optionally with bone threads, and an inner cannula.

Clause 17: The method of clause 16, wherein the implant is configured to move along the guide wire, and wherein the guide wire passes through the inner cannula of the implant.

Clause 18: The method of clause 16, wherein the implant further comprises an upper socket, and wherein the upper socket is configured to interface with a driver instrument for implant delivery.

Clause 19: The method of clause 13, wherein one or more components of the implant comprises a biocompatible material, stainless steel, titanium alloy, allograft bone, xenograft bone, synthetic bone material, medical polymers, polyetheretherketone, carbon fiber, polyetheretherketone reinforced with carbon fiber, and/or a bioresorbable material.

Clause 20: The method of clause 13, further comprising localizing a target facet joint using an imaging modality.

Clause 21: A system for ablation of a nerve comprising the device of any one of clauses 1 to 11 and an implant, wherein the implant is capable of blocking, retarding, modulating or quieting nerve growth or pain transmission from the medial branch nerve.

Clause 22: A kit for ablation of a nerve comprising the device of any one of clauses 1 to 11 and an implant, wherein the implant is capable of blocking retarding, modulating or quieting nerve growth or pain transmission from the medial branch nerve.

Clause 23: A system for ablation of a nerve comprising the device of any one of the clauses 1 to 11 and an implant, wherein the device and related instruments are configured to interface with a robotic system for anatomically targeting and/or application of a nerve ablation therapy.

Clause 24: A device for ablation of a nerve, comprising: a reamer tool, comprising a plurality of cutting flutes; a central axis, wherein the device is capable of rotating around the central axis; a shaft; and a coring tip, comprising a proximal end that connects with the reamer tool, a distal end, and a cylindrical wall connecting the proximal end and the distal end, wherein the cylindrical wall defines an inner biopsy chamber, and wherein the inner biopsy chamber has an opening at the distal end.

Clause 25: The device of clause 24, wherein the coring tip comprises a central axis that is aligned with the central axis of the device.

Clause 26: The device of clause 24, wherein the device comprises a central cannula, wherein the central cannula passes through the device or portion of the device along the central axis.

Clause 27: The device of clause 24, wherein the device is configured to move along a guide wire, wherein the guide wire passes through the central cannula.

Clause 28: The device of any one of clauses 24 to 27, wherein the plurality of cutting flutes are arranged in a centrifugal fashion around the central axis of the device.

Clause 29: The device of any one of clauses 24 to 28, wherein one or more of the plurality of cutting flutes is convex-shaped.

Clause 30: The device of clause 24, wherein the coring tip is configured to remove a cylindrical sample of bone material.

Clause 31: The device of clause 30, wherein the coring tip comprises one or more side openings in the cylindrical wall to facilitate manual expulsion of the cylindrical sample of bone material from the coring tip upon removal of the device from a body.

Clause 32: The device of clause 24, wherein the coring tip has a wall thickness that is less than ⅛ the diameter of the cylindrical sample of bone material which is removed in relation to the volume of bone defect created by the use of the device.

Clause 33: The device of clause 24, wherein the coring tip comprises a smooth outer surface to facilitate low friction rotation of the device during usage.

Clause 34: The device of clause 24, wherein the coring tip comprises a sharp distal edge to facilitate cutting of the distal edge of the device into bone tissue during usage.

Clause 35: The device of clause 34, wherein the coring tip comprises cutting teeth along the distal edge to facilitate cutting of hard bony material.

Clause 36: The device of clause 34, wherein the outer surface of the coring tip comprises a threaded profile to facilitate rotatable entry into a bone.

Clause 37: The device of any one of clauses 24 to 36, one or more components of the device comprises a biocompatible material, stainless steel, titanium alloy, cobalt chromium and/or medical grade plastics.

Clause 38: The device of any one of clauses 24 to 37, wherein the shaft is attached to a handle, and wherein the handle is capable of manipulating and controlling position of the device and applying rotational motion to the device.

Clause 39: An implant for blocking nerve growth, comprising: a cylindrical shaft with a long axis aligned with the center of the cylindrical shaft with said cylindrical shaft configured to be anchored into tissue; and an upper socket, wherein the upper socket is configured to interface with a driver instrument for implant delivery.

Clause 40: The implant of clause 39, wherein the tissue comprises bone tissue and/or soft tissue.

Clause 41: The implant of clause 39 or 40, wherein the cylindrical shaft comprises an outer threaded surface to facilitate anchorage into tissue.

Clause 42: The implant of any one of clauses 39 to 41, wherein the implant comprises a central cannula along the long axis, wherein the central cannula allows passage of a guide wire for placement of the implant.

Clause 43: The implant of clause 42, wherein the implant comprises a first material within the central cannula, and wherein the first material remains within the central cannula after removal of the guide wire.

Clause 44: The implant of clause 39, wherein the implant comprises one or more openings along the outer surface, and wherein the one or more openings are configured to contain a second material.

Clause 45: The implant of clause 44, wherein the one or more openings are at an angle to the long axis of the implant.

Clause 46: The implant of clause 44, wherein the one or more openings are configured to traverse the implant either along the long axis of the implant or at an angle to the long axis of the implant or both.

Clause 47: The implant of clause 44, wherein the second material directly or indirectly contacts tissues surrounding the implant after placement.

Clause 48: The implant of clause 44, wherein the second material comprises a bone material, a porous ceramic, a bioactive glass, polymeric matrix, a sponge, a foam, a growth factor, a pharmacologic agent, and/or a cytokine.

Clause 49: The implant of clause 44, wherein the second material comprises pharmacologic agents, medications, biologic molecules, nerve growth modulating agents, anti-inflammatory agents, growth factors, cytokines, and/or pain blocking or pain-modulating agents.

Clause 50: The implant of any one of clauses 39 to 49 wherein the implant comprises a base material, with or without a coating material.

Clause 51: The implant of clause 50, wherein the base material comprises a biocompatible material, stainless steel, titanium alloy, cobalt chromium, medical grade plastics, a medical grade polymeric compound, a bioresorbable material, and/or a bone material from allograft, xenograft, or synthetically manufactured bone.

Clause 52: The implant of clause 48, wherein the bone material is derived from bone of a subject that receives the implant or from bone of a donor, wherein the donor is alive or deceased or from another species or from synthetically manufactured bone.

Clause 53: The implant of any one of clauses 50, wherein the coating material comprises anti-nerve-growth coating, anti-inflammatory coating, antimicrobial coating, antibacterial coating, antithrombotic coating, anticoagulant coating, hydrophilic coating, hydrophobic coating, drug-eluting coating, biocompatible coating, bioactive coating, growth factor containing, cytokine containing, lubricious coating, anti-adhesion coating, anti-fouling coating, and/or radiopaque coating.

Clause 54: A method for blocking nerve growth, comprising the steps of: disrupting a nerve by producing a cavity in bone tissue; and delivering the implant of any one of clauses 39 to 53 to the cavity.

Clause 55: The method of clause 54, wherein the nerve is a medial branch nerve of the dorsal ramus.

Clause 56: A method for ablation of a nerve, comprising the steps of: docking the device of any one of clauses 1 to 11 or 24 to 38 against a vertebra at a base of a transverse process of the vertebrae; rotating the docked device to cause the plurality of cutting flutes to contact with bone tissue at the base of the transverse process of the vertebrae; producing a cavity in the bone tissue by the rotating of the docked device, wherein tissue in the cavity is removed; and disrupting a medial branch nerve by producing the cavity in the bone tissue.

Clause 57: The method of clause 56, further comprising delivering an implant to the cavity via a guide wire, wherein the implant comprises: an outer surface, wherein a portion of the outer surface is threaded; a hollow inner channel; and an upper socket, wherein the upper socket is configured to interface with a driver instrument for implant delivery.

Clause 58: The method of clause 57, wherein the outer surface comprises one or more openings that are connected to the hollow inner channel.

Clause 59: The method of any one of clauses 57 to 58, further comprising a core that is positioned within the hollow inner channel, wherein at least part of the core is exposed through the one or more openings.

Clause 60: The method of clause 59, wherein the core comprises a bone material, a porous ceramic, a bioactive glass, a sponge, and/or a foam.

Clause 61: The method of any one of clauses 57 to 60, wherein the implant comprises a base material and a coating material.

Clause 62: The method of clause 61, wherein the base material comprises a biocompatible material, stainless steel, titanium alloy, cobalt chromium, medical grade plastics, a bioresorbable material, and/or a bone material of allograft, xenograft, or synthetically manufactured bone.

Clause 63: The method of clause 62, wherein the bone material is derived from bone of a subject that receives the implant or from bone of a donor, wherein the donor is alive or deceased or from another species or from synthetically manufactured bone.

Clause 64: The method of any one of clauses 61 to 63, wherein the coating material comprises anti-nerve-growth coating, anti-inflammatory coating, antimicrobial coating, antibacterial coating, antithrombotic coating, anticoagulant coating, hydrophilic coating, hydrophobic coating, drug-eluting coating, biocompatible coating, bioactive coating, growth factor containing, cytokine containing, lubricious coating, anti-adhesion coating, anti-fouling coating, and/or radiopaque coating.

Clause 65: A method for ablation of a nerve, comprising the steps of: docking the device of any one of clauses 24 to 38 against a vertebra at a base of a transverse process of the vertebrae; rotating the docked device to cause the plurality of cutting flutes to contact with bone tissue at the base of the transverse process of the vertebrae; producing a cavity in the bone tissue by the rotating of the docked device, wherein tissue in the cavity is removed by the coring tip and captured in the inner biopsy chamber of the coring tip; and disrupting a medial branch nerve by producing the cavity in the bone tissue.

Clause 66: The method of clause 65, further comprising: ejecting the removed tissue from the coring tip; loading the removed tissue into an implant; and delivering the implant to the cavity via a guide wire.

Clause 67: The method of clause 65, wherein the implant comprises: an outer surface, wherein a portion of the outer surface is threaded; a hollow inner channel; and an upper socket, wherein the upper socket is configured to interface with a driver instrument for implant delivery; wherein the outer surface comprises one or more openings that are connected to the hollow inner channel.

Clause 68: The method of clause 67, wherein the removed tissue is positioned within the hollow inner channel of the implant, wherein at least part of the removed tissue is exposed through the one or more openings.

### EXAMPLES

The following examples are included to demonstrate exemplary embodiments of the disclosure. It should be appreciated by those of skill in the art that the techniques disclosed in the examples which follow represent techniques discovered by the inventor(s) to function well in the practice of the disclosure, and thus can be considered to constitute exemplary modes for its practice. However, those of skill in the art should, in light of the present disclosure, appreciate that many changes can be made in the specific embodiments which are disclosed and still obtain a like or similar result without departing from the spirit and scope of the disclosure. For example, the embodiments may be of various sizes, lengths, measures, and/or dimensions, including but not limited to the exemplary marks in the figures.

As shown in FIG. 1, FIG. 1A illustrates a device that includes a reamer tool that has a trocar tip, a plurality of cutting flutes, and a shaft. The shaft of the device A110 may be attached to or connected with the handle A120. The handle A120 may bear depth marks (e.g., 4, 6, 8, and 10 cm) on its outer surface. A130 illustrates an indicator tape, which may have one or more colors. Indicator tape A130 may fit onto the outer surface of the handle A120.

FIG. 1B illustrates a device with a reamer tool that has a central cannula, a plurality of cutting flutes, and a shaft. The shaft of the device B110 may be attached to or connected with the handle B120. The handle B120 may bear depth marks (e.g., 4, 6, 8, and 10 cm) on its outer surface. B130 illustrates an indicator tape, which may have one or more colors. Indicator tape B130 may fit onto the outer surface of the handle B120. FIG. 1C illustrates an assembly of the device B110 and the handle B120. FIG. 1D illustrates a longitudinal section view of the assembly in FIG. 1C, showing the central cannula of the device B110 is connected with an inner lumen of the handle in FIG. 1C (or handle B120), both of which form a continuous channel for a guide wire to pass through.

FIGS. 1E-1I illustrate different views of an implant that is a cannulated cylindrical screw. This implant has an outer threaded surface with bone threads and an inner passage (or cannulation) that could be used to deliver the implant over a guide wire. The implant also has an upper socket to interface with a driver instrument for implant delivery. FIG. 1H illustrates a longitudinal section view of the implant illustrated in FIGS. 1E-1G, showing an inside view of the inner passage (or cannulation), bone threads, and the upper socket. The upper socket may have inner threads that can connect with the driver instrument. FIG. 1I illustrates a cross section view of the upper socket of the implant illustrated in FIGS. 1E-1H.

As shown in FIG. 2, FIG. 2A illustrates an assembly that has device A210 with a reamer tool that has a coring tip, a plurality of cutting flutes, and a shaft. Device A210 is further illustrated in FIGS. 2B-2E. The shaft of device A210 may be attached to or connected with handle A230. Handle A230 may bear depth marks (e.g., 4, 6, 8, and 10 cm) on its outer surface. A220 illustrates an indicator tape, which may have one or more colors. The indicator tape may fit onto the outer surface of handle A230.

FIGS. 2B and 2D illustrate different views of device A210 that includes a reamer tool that has a coring tip, a plurality of cutting flutes, and a shaft. FIG. 2C is a top view of the reamer tool showing an opening of an inner biopsy chamber at the distal end of the reamer tool. FIG. 2C also shows plurality of cutting flutes that are arranged in a centrifugal fashion around the coring tip of the reamer tool. FIG. 2E illustrates a longitudinal section view of device A210, showing that the coring tip has a proximal end that connects with the reamer tool, a distal end, and a cylindrical wall connecting the proximal end and the distal end, where the cylindrical wall defines an inner biopsy chamber, and where the inner biopsy chamber has an opening at the distal end. FIG. 2E also illustrates that device A210 may include a central cannula, where the central cannula may pass through the device or portion of the device along the central axis.

FIG. 2G illustrates a longitudinal section view of the assembly in FIG. 2F, showing the central cannula of the device in FIG. 2F is connected with an inner lumen of the handle in FIG. 2H (or FIG. 2F), both of which form a continuous channel for a guide wire to pass through.

FIGS. 2I-2L illustrate different views of an implant that is a cannulated cylindrical or cone-shaped screw, with one or more openings along the outer surface (e.g., shown in J210 and K210). This implant has an outer threaded surface with bone threads and an inner passage (or cannulation) that could be used to deliver the implant over a guide wire. The implant also has an upper socket to interface with a driver instrument (e.g., shown in FIG. 2M-2O) for implant delivery. FIG. 2I illustrates a longitudinal section view of the implant illustrated in FIGS. 2I-2L, showing an inside view of the inner passage (or cannulation), bone threads, and the upper socket. The upper socket may have inner threads that can connect with the driver instrument. FIG. 2L illustrates a cross section view of the upper socket of the implant illustrated in FIGS. 2I-2L.

FIGS. 2M-2O illustrate different views of driver instrument that can receive and deliver an implant (e.g., shown in FIGS. 2I-2L). FIG. 2M illustrates an assembly that has an implant and a driver instrument. The driver instrument may bear depth marks (e.g., 1, 3, 5, 7, and 9 cm) on its outer surface. FIG. 2N is a longitudinal section view of the driver instrument that has implant N210 loaded at its distal end through the upper socket of the implant. The upper socket may have inner threads that can connect with the driver instrument. The driver instrument has an outer wall N220 and hollow inner shaft N230. The inner passage (or cannulation) of implant N210 is connected with hollow inner shaft N230 of the driver instrument, both of which form a continuous channel for a guide wire to pass through. The driver instrument, at its proximal end, has twisting tip N240 that is connected to hollow inner shaft N230. A user may twist or manipulate twisting tip N240 to rotate hollow inner shaft N230, which in turn will cause rotation of implant N210 at the user's control.

All of the devices and/or methods disclosed and claimed herein can be made and executed without undue experimentation in light of the present disclosure. While the devices and methods of this disclosure have been described in terms of exemplary embodiments, it will be apparent to those of skill in the art that variations may be applied to the methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

What is claimed is:

1. A device for ablation of a nerve, comprising:
a reamer tool, comprising a plurality of cutting flutes;
a central axis, wherein the device is capable of rotating around the central axis;
a shaft; and
a coring tip, comprising a proximal end that connects with the reamer tool, a distal end, and a cylindrical wall connecting the proximal end and the distal end, wherein the cylindrical wall defines an inner biopsy chamber, and wherein the inner biopsy chamber has an opening at the distal end.

2. The device of claim 1, wherein the coring tip comprises a central axis that is aligned with the central axis of the device.

3. The device of claim 1, wherein the device comprises a central cannula, wherein the central cannula passes through the device or portion of the device along the central axis.

4. The device of claim 1, wherein the device is configured to move along a guide wire, wherein the guide wire passes through the central cannula.

5. The device of claim 1, wherein the plurality of cutting flutes are arranged in a centrifugal fashion around the central axis of the device.

6. The device of claim 1, wherein one or more of the plurality of cutting flutes is convex-shaped.

7. The device of claim 1, wherein the coring tip is configured to remove a cylindrical sample of bone material.

8. The device of claim 7, wherein the coring tip comprises one or more side openings in the cylindrical wall to facilitate manual expulsion of the cylindrical sample of bone material from the coring tip upon removal of the device from a body.

9. The device of claim 1, wherein the coring tip has a wall thickness that is less than ⅛ the diameter of the cylindrical sample of bone material which is removed in relation to the volume of bone defect created by the use of the device.

10. The device of claim 1, wherein the coring tip comprises a smooth outer surface to facilitate low friction rotation of the device during usage.

11. The device of claim 1, wherein the coring tip comprises a sharp distal edge to facilitate cutting of the distal edge of the device into bone tissue during usage.

12. The device of claim 11, wherein the coring tip comprises cutting teeth along the distal edge to facilitate cutting of hard bony material.

13. The device of claim 11, wherein the outer surface of the coring tip comprises a threaded profile to facilitate rotatable entry into a bone.

14. The device of claim 1, one or more components of the device comprises a biocompatible material, stainless steel, titanium alloy, cobalt chromium and/or medical grade plastics.

15. The device of claim 1, wherein the shaft is attached to a handle, and wherein the handle is capable of manipulating and controlling position of the device and applying rotational motion to the device.

16. A method for ablation of a nerve, comprising the steps of:
docking a device against a vertebra at a base of a transverse process of the vertebrae, wherein the device comprises:
a reamer tool, comprising a plurality of cutting flutes,
a shaft, and
a coring tip, comprising a proximal end that connects with the reamer tool, a distal end, and a cylindrical wall connecting the proximal end and the distal end, wherein the cylindrical wall defines an inner biopsy chamber, and wherein the inner biopsy chamber has an opening at the distal end;
rotating the docked device to cause the plurality of cutting flutes to contact with bone tissue at the base of the transverse process of the vertebrae;
producing a cavity in the bone tissue by the rotating of the docked device, wherein tissue in the cavity is removed by the coring tip and captured in the inner biopsy chamber of the coring tip; and
disrupting a medial branch nerve by producing the cavity in the bone tissue.

17. The method of claim 16, further comprising:
ejecting the removed tissue from the coring tip;
loading the removed tissue into an implant; and
delivering the implant to the cavity via a guide wire.

18. The method of claim 16, wherein the implant comprises:
an outer surface, wherein a portion of the outer surface is threaded;
a hollow inner channel; and
an upper socket, wherein the upper socket is configured to interface with a driver instrument for implant delivery;
wherein the outer surface comprises one or more openings that are connected to the hollow inner channel.

19. The method of claim 18, wherein the removed tissue is positioned within the hollow inner channel of the implant, wherein at least part of the removed tissue is exposed through the one or more openings.

20. The method of claim 16, wherein the coring tip is configured to remove a cylindrical sample of bone material.
